(19) United States
(12) Patent Application Publication
(10) Pub. No.: US 2023/0158292 A1
(43) Pub. Date: May 25, 2023

(54) Non-invasive electrical stimulation wearable system for targeting the olfactory regions

(71) Applicants: Yusuf Ozgur Cakmak, Dunedin (NZ); Abhishek Datta, Watchung, NJ (US); Kamran Nazim, Somerset, NJ (US); Chris Thomas, Queens Village, NY (US)

(72) Inventors: Yusuf Ozgur Cakmak, Dunedin (NZ); Abhishek Datta, Watchung, NJ (US); Kamran Nazim, Somerset, NJ (US); Chris Thomas, Queens Village, NY (US)

(21) Appl. No.: 17/512,619

(22) Filed: Oct. 27, 2021

(51) Int. Cl. A61N 1/04; A61N 1/36

(52) U.S. Cl. CPC A61N 1/0408; A61N 1/36025; A61N 1/36031

(57) ABSTRACT

A system and method for non-invasively applying electrical stimulation to target the olfactory bulb and olfactory mucosa. Current is delivered through an optimal montage of electrodes positioned on the lower forehead, nose bridge, and neck posterior for suppressing the progression of disorders or alleviating symptoms caused by olfactory dysfunction such as Alzheimer’s Disease, Parkinson’s Disease, Loss of Consciousness, etc. Electrode placements are proposed depending on application- for instance using only hair free zones for stimulation. This invention was made with government support under Grant R44NS092144 awarded by the National Institutes of Health. The government has certain rights in the invention.

### REFERENCES CITED

### Cross Reference to Related Applications

This application claims benefit to provisional application No. 63106129, filed on Oct. 27, 2020

### OTHER PUBLICATIONS

Alves, J., Petrosyan, A., and Magalhães, R. (2014). Olfactory dysfunction in dementia. World J. Clin. Cases 2, 661-667. doi: 10.12998/wjcc.v2.i 11.661
Arzi, A., Rozenkrantz, L., Gorodisky, L., Rozenkrantz, D., Holtzman, Y., Ravia, A., et al. (2020). Olfactory sniffing signals consciousness in unresponsive patients with brain injuries. Nature 581, 428-433. doi: 10.1038/s41586-020-2245-5
Q16 Bikson, M., and Datta, A. (2012). Guidelines for precise and accurate computational models of tDCS. Brain Stimul. 5, 430-431. doi: 10.1016/j.brs. 2011.06.001
Braak, H., and Braak, E. (1991). Neuropathological stageing of Alzheimer-related changes. Acta Neuropathol. 82, 239-259. doi: 10.1007/BF00308809
Braak, H., Del Tredici, K., Rüb, U., de Vos, R. A., Jansen Steur, E. N., and Braak, E. (2003). Staging of brain pathology related to sporadic Parkinson’s disease. Neurobiol. Aging 24, 197-211. doi: 10.1016/s0197-4580(02)00065-9
Criscuolo, C., Fontebasso, V., Middei, S., Stazi, M., Ammassari-Teule, M., Yan, S. S., et al. (2017). Entorhinal cortex dysfunction can be rescued by inhibition of microglial RAGE in an Alzheimer’s disease mouse model. Sci. Rep. 7:42370. doi: 10.1038/srep42370
Datta, A., Dmochowski, J. P., Guleyupoglu, B., Bikson, M., and Fregni, F. (2013). Cranial electrotherapy stimulation and transcranial pulsed current stimulation: a computer based high-resolution modeling study. NeuroImage 65, 280-287. doi: 10.1016/j.neuroimage.2012.09.062
Datta, A., Elwassif, M., Battaglia, F., and Bikson, M. (2008). Transcranial current stimulation focality using disc and ring electrode configurations: FEM analysis. J. Neural Eng. 5, 163-174. doi: 10.1088/1741-2560/5/2/007
Datta, A., Truong, D., Minhas, P., Parra, L. C., and Bikson, M. (2012). Inter-individual variation during transcranial direct current stimulation and normalization of dose using MRI-Derived computational models. Front. Psychiatry 3:91. doi: 10.3389/fpsyt.2012.00091
Desikan, R. S., McEvoy, L. K., Thompson, W. K., Holland, D., Brewer, J. B., Aisen, P. S., et al. (2012). Amyloid-β-associated clinical decline occurs only in the presence of elevated P-tau. Arch. Neurol. 69, 709-713. doi: 10.1001/archneurol. 2011.3354
Doty, R. L. (2012). Olfactory dysfunction in Parkinson disease. Nat. Rev. Neurol. 8, 329-339. doi: 10.1038/nrneurol.2012.80
Doty, R. L., and Kamath, V. (2014). The influences of age on olfaction: a review. Front. Psychol. 5:20. doi: 10.3389/fpsyg.2014.00020
Duff, K., McCaffrey, R. J., and Solomon, G. S. (2002). The pocket smell test: successfully discriminating probable Alzheimer’s dementia from vascular dementia and major depression. J. Neuropsychiatry Clin. Neurosci. 14, 197-201. doi: 10.1176/jnp.14.2.197
Edwards, D., Cortes, M., Datta, A., Minhas, P., Wassermann, E. M., and Bikson, M. (2013). Physiological and modeling evidence for focal transcranial electrical brain stimulation in humans: a basis for high-definition tDCS. Neuroimage 74, 266-275. doi: 10.1016/j.neuroimage.2013.01.042
Jiang H., Schuele S., Rosenow J., et al., Theta Oscillations Rapidly Convey Odor-Specific Content in Human Piriform Cortex. 2017, Neuron 94, 207-219
Cakmak YO, Nazim K, Thomas C, Datta A. Optimized Electrode Placements for Non-invasive Electrical Stimulation of the Olfactory Bulb and Olfactory Mucosa. Front Neurosci. 2020 Nov 12;14:581503.

### A NON-INVASIVE ELECTRICAL STIMULATION WEARABLE SYSTEM FOR TARGETING THE OLFACTORY REGIONS

Background of the Invention: The field of the present invention is related to non-invasive modulation of the olfactory regions to achieve a therapeutic result. The olfactory system is known to be dysfunctional in the early stages of Parkinson’s disease and Alzheimer’s disease (Rezek, 1987; Mesholam et al., 1998; Duff et al., 2002; Motomura and Tomota, 2006; Fusetti et al., Q12 2010; Doty, 2012; Alves et al., 2014; Doty and Kamath, 2014). Additionally, intact olfactory function can be a key role player for regaining consciousness after brain injuries. Modulation of the olfactory regions has been attempted successfully with electrical stimulation over the years, but never in a non-invasive fashion such as the one used in this invention. Recent research has shown that 100% (specificity) of patients who respond to olfactory stimuli in unresponsive states or low levels of consciousness regained consciousness (Arzi et al., 2020). There have been attempts to deliver electrical stimulation directly to the olfactory epithelium via positioning the stimulation electrodes as close as possible to the “target.” These placements all involved electrode insertion through the nostril, but studies employed a range of stimulation dose, application precision, and desired olfactory targets. Straschill et al. (1983) performed stimulation by an electrode attached to a rhinoscope and delivered 2 mA/0.5 ms pulses. Ishimaru et al. (1997, 2002). Several studies from 1997 to the most recent study in 2002, used a bipolar stimulating electrode (with no indication of guiding mechanism used) and also delivered 2 mA/0.5 ms pulses. Weiss et al. (2016) and more recently Holbrook et al. (2019) both used an electrode placed with endoscopic guidance. Weiss tested a range of stimulation parameters applied at currents ranging between 50 and 800 µA targeting the ventral surface of the middle turbinate. Holbrook et al. (2019) used constant square wave pulses with gradually increasing intensity from 1 to 20 mA and importantly targeted the OB through the thin bone of the cribriform plate. In addition, there have been efforts to affect olfactory function by using subdural electrodes targeting frontal lobes proximal to the olfactory bulb (Kumar et al., 2012). This invention is the first to modulate olfactory function with 80-Hz of non-invasive electrical stimulation of the auricular vagus nerve in humans it is the only known means of delivering optimized electrical stimulation to the olfactory region in a non-invasive fashion, and is simpler, easier, and less cumbersome than the existing methods (Cakmak 2020). Additional benefits can be achieved by applying stimulation in closed-loop systems with physiological sensors such as electromyography (EMG), electroencephalography (EEG), infra-red sensing, artificial intelligence (AI) based image recognition, or electrocardiography (ECG). Sometimes, additional benefit can be achieved by synchronizing stimulation with a segment of the respiration cycle, preferably at the end of the inspiration phase to mimic the physiological firing of the olfactory neurons (Jiang 2017).

Summary of the Invention: In one aspect of the invention, devices and methods are described to stimulate the olfactory regions in a patient by utilizing non invasive electrical stimulation. In particular, the disclosed devices deliver current through strategically placed electrodes positioned on the lower forehead, nose bridge, and/or neck posterior of the patient.

In another aspect of the invention an ideal module for stimulating the olfactory epithelium involves placing non invasive electrodes on each side of the nose bridge and placing two non invasive electrodes on the upper posterior region of the scalp.

In another aspect of the invention, an ideal module for stimulating the hippocampal and parahippocampal cortex involves placing non-invasive electrodes on each side of the nose bridge and two electrodes on the upper posterior region of the scalp.

In another aspect of the invention, an ideal module for stimulating the hippocampal and parahippocampal cortex involves placing 3 non-invasive electrodes on the lower forehead, one electrode on each side of the nose bridge, and two electrodes on the neck posterior.

In another aspect of the invention stimulation is delivered in closed-loop systems with physiological sensors including but not limited to one or more of the following: electromyography, electroencephalography, infra-red sensing, artificial intelligence based image recognition, or electrocardiography.

In another aspect of the invention stimulation is synchronised with a particular segment of the respiration cycle, preferably at the end of the inspiration phase to mimic the physiological firing of the olfactory neurons.

### BRIEF DESCRIPTION OF THE DRAWINGS

For the purposes of illustrating the various aspects of the invention, there are shown in the drawings forms that are presently preferred, it being understood, however, that the invention is not limited by or to the precise data, methodologies, arrangements a

FIG. 1 is a frontal view of a first montage with two disc shaped electrodes (10-11) positioned on either side of the nose bridge.

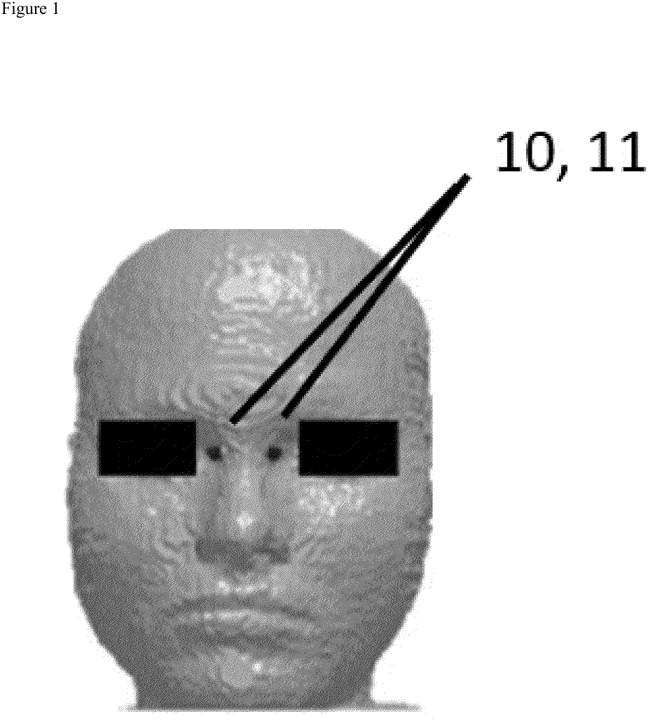

FIG. 2 is a back view of the first montage with two oval disc shaped electrodes (20-21) positioned on the posterior side of the scalp.

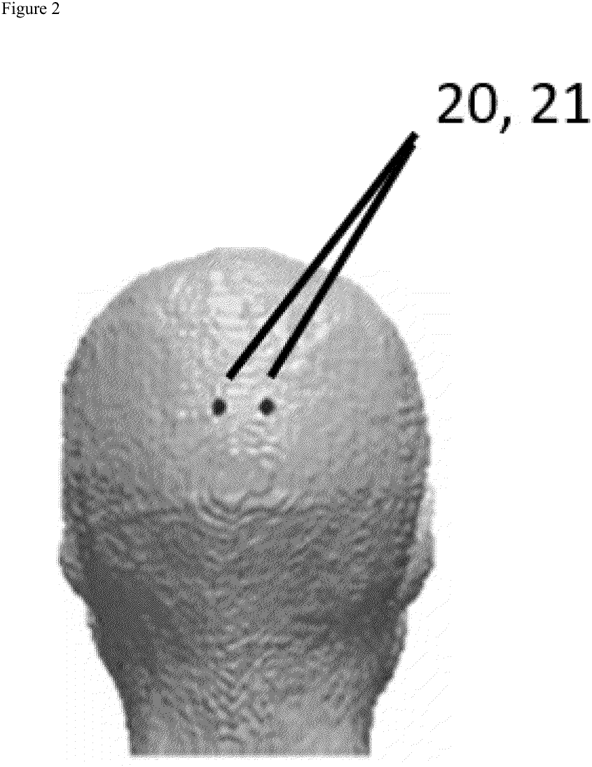

FIG. 3 is a frontal view of a second montage with three small circular electrodes (30-32) positioned in the center of the lower forehead and two small circular electrodes (33-34) positioned on the nose bridge.

FIG. 4 is a back view of the second montage with two disc shaped electrodes (40-41) positioned on the neck posterior

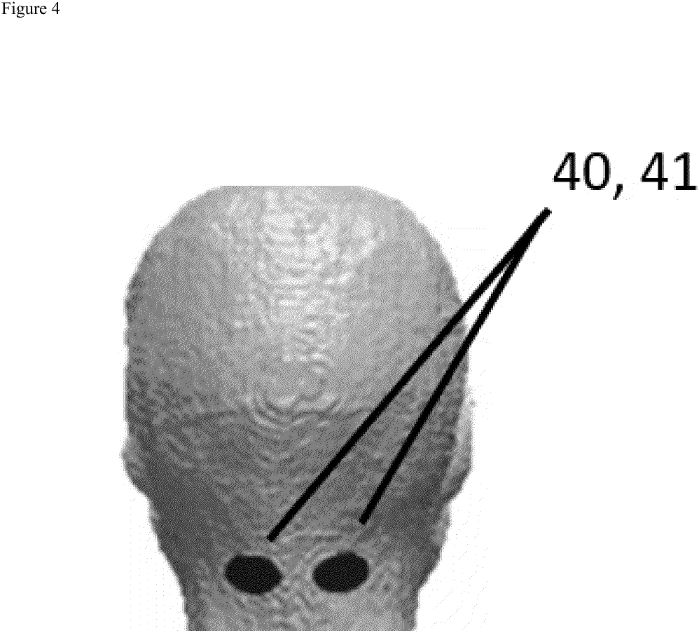

FIG. 5 is a frontal view of a third montage with one small circular electrode (50) positioned in the center of the lower forehead and two small circular electrodes (51-52) positioned on the nose bridge.

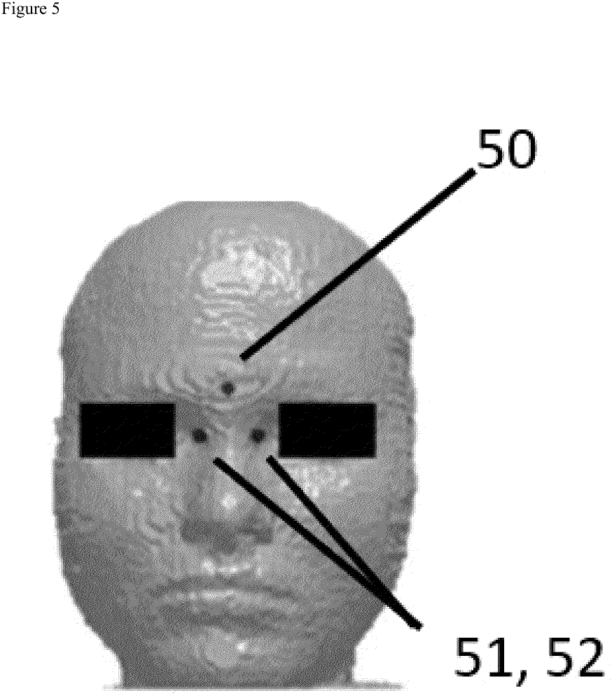

FIG. 6 is a back view of the third montage with two oval shaped electrodes (60-61) positioned on the neck posterior.

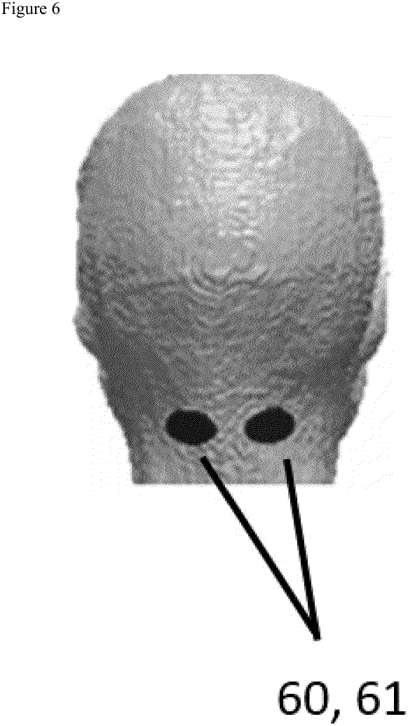

FIG. 7 is a frontal-side view of a device for delivering stimulation to the olfactory region. One electrode (70) is positioned on the neck posterior. One electrode (71) is positioned in the center of the lower forehead. Two electrodes (72-73) are positioned on the nose bridge.

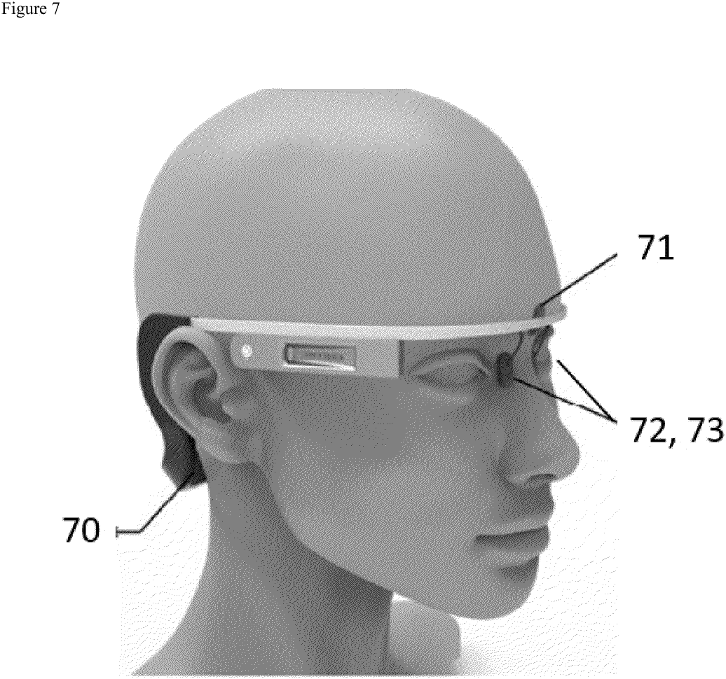

FIG. 8 is a side view of the device for delivering stimulation to the olfactory region. A Camera (80) is housed in a forehead portion of the device.

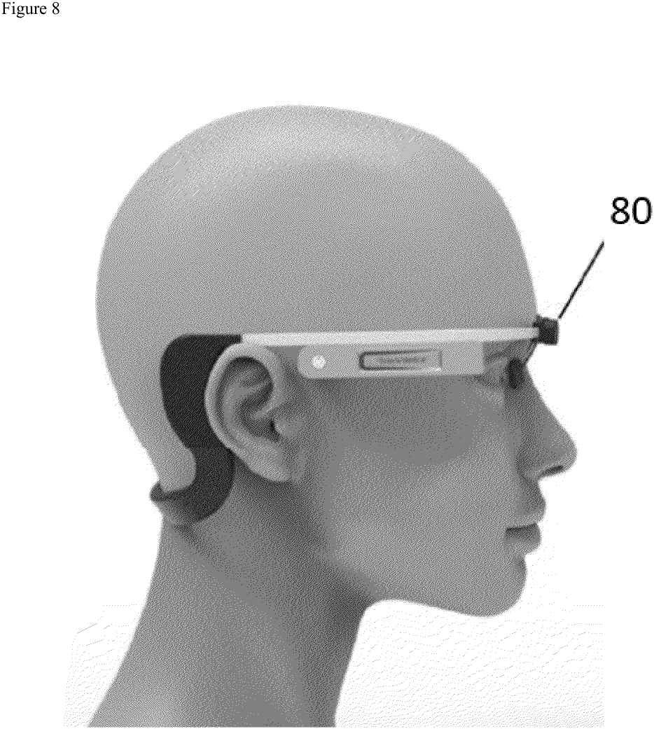

### DETAILED DESCRIPTION OF THE PREFERRED EMBODIMENTS

In the present invention, energy is transmitted non-invasively to a patient to modulate their olfactory region. The invention is particularly useful for producing applied electrical impulses that interact with the olfactory region to treat Alzheimer’s disease, Parkinson’s disease, and other neurological disorders. In particular, the present disclosure describes devices and methods to stimulate the olfactory region, non invasively by strategically placing electrodes on the patient’s forehead, neck, and/or nose bridge.

There is a long-felt but unsolved need to non-invasively, and painlessly modulate the olfactory region to treat neurological disorders. As discussed below, this is evidenced by others who have tried to invasively stimulate the olfactory region by inserting electrodes through the patient’s nostrils. Straschill et al. (1983) performed stimulation by an electrode attached to a rhinoscope and delivered 2 mA/0.5 ms pulses. Ishimaru et al. (1997, 2002), in several studies from 1997 to the most recent study in 2002, used a bipolar stimulating electrode and also delivered 2 mA/0.5 ms pulses. Weiss et al. (2016) and more recently Holbrook et al. (2019) both used an electrode placed with endoscopic guidance. While all these approaches were attempts at modulating the olfactory region, non were non-invasive.

Potential advantages of non-invasive methods and devices relative to comparable invasive procedures are as follows. The patient may be more psychologically prepared to experience a procedure that is non-invasive and may therefore be more cooperative, resulting in a better outcome. Non-invasive procedures may avoid damage of biological tissues, such as those due to bleeding or infection. In cases particularly involving the attachment of electrodes, non-invasive methods have less of a tendency for breakage of leads, and the electrodes can be easily repositioned if necessary.

The applicant discovered the disclosed devices and methods over the course of experimentation to determine the most promising placement of electrodes to stimulate the olfactory system including the olfactory epithelium, olfactory bulb, and entorhinal cortex. To do this they simulated the current flow distribution in the olfactory system, including the olfactory epithelium, olfactory bulb, and entorhinal cortex as well as in the entire brain. They developed an ultra-high-resolution model based on a 0.5 mm isotropic resolution dataset which was necessary to resolve the tiny structures of interest in this study. They determined induced surface and cross- sectional electric field maps for the entire brain for each of the electrode placements considered. They also compared focality relative to an electric field threshold value to enable a quantitative comparison. Furthermore, current flows in the olfactory epithelium, olfactory bulb, basal ganglia, and hippocampus were individually analyzed and additional metrics of polarization considered for a subset of the configurations.

The ultra-high-resolution head and neck model (MIDA: multimodal imaging-based detailed anatomical) available through the IT’IS Foundation was used in this study (Iacono et al., 2015). The MIDA model was merged with a cropped version of Duke (Visible Human Project) to extend the model until the level of the chest. In order to do so, the Duke model was first cropped at the level of the chest and then imported into the same anatomical space as the MIDA model. The Duke model geometry was then aligned with the neck region of the MIDA model and the contact interfaces modified by applying appropriate filters to minimize any abrupt transition between the two models. This process ensured a simplified geometry at the merged sections leading to a simpler mesh.

The nifti (.nil) color masks from the MIDA model were first processed in MATLAB to re-create segmentation masks based on intensity values. These masks were then imported into Simpleware (Synopsys Ltd., CA, United States), and any errors in continuity and anatomical details were manually corrected for Datta et al. (2012); Haberbosch et al. (2019). Masks with similar electrical conductivities were then merged to a single compartment excluding the regions of interest (OB and OE) in order to perform detailed individual current flow analysis through them. The OE was extracted from the upper third of the inner mucosa region of the MIDA model using the 3D editing tool in Simpleware. The area was determined by using a combination of visual references as a guide (Purves et al., 2001; Nakao et al., 2015). The stimulation electrodes and the conductive media (gel) were created as computer-aided design (CAD) models (STL files) having either circular or oval disk shapes and were positioned interactively within the image dataset. The circular disks were smaller (6-mm diameter) while the oval disks bigger (long axis = 25 mm; short axis = 20 mm). All electrode disks had a thickness of approximately 1 mm and were modeled as conductors with the conductivity of copper. The thickness of the gel disks were approximately 1 mm and were assumed to have the conductivity of a typical conductive gel used for electrical stimulation applications.

The following six novel montages were simulated:
1. Nose bridge + upper posterior (Montage 1): Two circular disks were placed on the immediate right and left of the nose bridge and two oval-shaped disks placed at the back of the head. The back electrodes were positioned corresponding 1 cm to the left and right of the midline at the level of POz (location corresponding to the 10-10 EEG layout).
2. Lower forehead and nose bridge + neck posterior (Montage 2): Three circular disks positioned on the lower forehead (slightly above the eyebrow). The disks were placed 1.5 cm apart from each other, about 1 cm above the nasion, with the central electrode along the midline. The nose bridge disks were placed similarly to Montage 1. This combination was paired with two oval-shaped electrodes positioned about 4.5 cm below the level of Iz and spaced about 4 cm from each other. The posterior electrodes were also positioned symmetrically from the midline.
3. Lower forehead and nose bridge + neck posterior (Montage 3): Same as Montage 2 but with one electrode on the lower forehead positioned along the midline. This combination was paired with two oval-shaped electrodes positioned at a similar location to Montage 2.
4. Upper forehead + lower posterior (Montage 4): Two circular disks were positioned on the forehead with two circular disc electrodes positioned on the lower back of the head. The forehead electrodes were placed about 3 cm above the nasion and about 1.5 cm away from each other or 0.75 cm on either side of the midline right below Fpz. The lower posterior electrodes were placed about 1 cm left and right of the midline at the level of the inion.
5. Lower forehead + neck posterior (Montage 5): Three circular disks were positioned on the lower forehead (slightly above the eyebrow) with four oval-shaped disk electrodes positioned on the upper neck. The disks were placed 1.5 cm apart from each other, about 1 cm above the nasion, with the central electrode along the midline. The four oval-shaped electrodes were positioned about 4.5 cm below the level of Iz and spaced about 4 cm from each other. The posterior electrodes were also positioned symmetrically from the midline.
6. Lower forehead + behind ear (Montage 6): Three circular disks positioned similarly on the lower forehead as Montage 5 configuration and two electrodes behind the ear avoiding the hairline. The behind ear electrodes corresponded to P9 and P10 of the 10-10 EEG layout.

The integrated CAD files were converted to masks, and appropriate filters and Boolean operations applied to ensure no overlapping tissue masks. Adaptive meshes derived from the segmentation and the CAD masks are then created for finite element analysis in COMSOL Multiphysics (Burlington, MA, United States). The final models on an average comprised >30 million elements with >50 million degrees of freedom. The studies targeting the olfactory system have typically considered T = 0.5 ms duration pulses. A modification of the standard Laplace’s equation incorporating a reactive component to account for the frequency (spectral content) is appropriate to determine the induced EF:

$$▽ ⋅ \left(\text{σ} + \text{jωε}\right) ▽ \text{V=0 where ε is permittivity and ω is angular frequency} \text{.}$$

The corresponding Fourier magnitude spectrum of a 0.5-ms duration pulse indicates power concentrated from 0 to 2 kHz with 2 kHz reflecting the first zero crossing (⅟T). The consideration of tissue properties (conductivity and permittivity) at 1 kHz (half of the first zero crossing) reveals that the real component of equation dominates such that the reactive component can be ignored (Gabriel et al., 1996; Edwards et al., 2013). This results in a simplified standard Laplace’s equation:

$$▽ ⋅ ( \text{σ} ▽ \text{V} ) = 0$$

Furthermore, tissue conductivity properties at 1 kHz are not substantially different than 0 Hz and have been shown experimentally to not result in any scalp potential differences (Datta et al., 2013). Taken together, 0 Hz or DC conductivity values are therefore considered here. The boundary conditions used were as follows: (1) inward current flow = Jn (normal current density) applied to the exposed surface of all the anterior electrodes considered in the individual montages (nose bridge, upper forehead, and lower forehead), (2) ground applied to the exposed surface of all posterior electrodes considered in the individual montages (upper posterior, lower posterior, neck posterior, and behind ear), and (3) all other external surfaces treated as insulated. The current density corresponding to 1 mA exemplary total injected current was considered for all montages.

Electrical field (EF) magnitude plots on the cortical surface and on olfactory epithelium generated for each of the six novel montages. To facilitate a quantitative comparison, the inventors determined stimulation focality by percentage volume of olfactory epithelium subject to EF magnitude greater than EF threshold value (arbitrarily chosen range but expected for 1 mA intensity considered). For each of the selected optimal montages, surface EF plots were generated for the olfactory bulb and olfactory epithelium. In addition, cross-sectional EF plots were generated to visualize depth modulation.

Finally, in addition to EF, the inventors considered two other drivers of neuronal polarization along an exemplary axon in the olfactory epithelium (EF in the axon direction and the activating function) to further elucidate differences between the configurations with respect to these “driving functions” (Warman et al., 1992; Rubinstein, 1993; McIntyre and Grill, 1999). This exemplary axon orientation was simulated to mimic actual anatomical orientation. We note that since the OB primarily consists of neuron bodies, we do not consider any other driver of neuronal polarization in the OB other than the electric field (Datta et al., 2008).

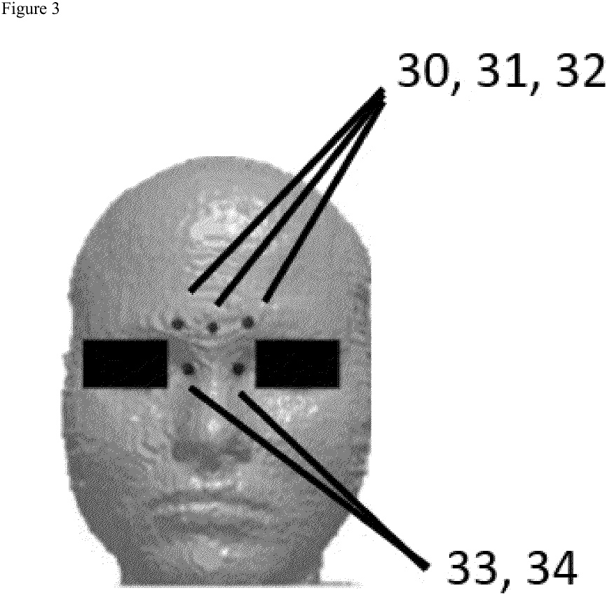

Brain current flow (electric field) was predicted using an FE model derived from an ultra-high-resolution dataset. For each one of the six initial candidate montages, the inventors first calculated the induced electric field magnitude on the brain surface. These plots allow a direct comparison of the relative surface focality and thereby screening for montages with higher focality for additional analysis. We observed that Montage 1,, Montage 2, and Montage 3 resulted in increased current flow in the olfactory epithelial regions. Each one of these montages comprise of electrodes positioned on either side of the nose bridge indicating that the proximity to the target ROI plays a predominant role in increased focality. Further, the OE volume percentage plots exceeding a particular EF threshold indicate that for Montages 1, 2, and 3, greater than 75% of the volume is subject to a value >1 V/m (FIG. 3). Montage 2 resulted in the lowest volume percentage > 1 V/m (~40%) confirming the observation from the surface plot showing reduced EF in the olfactory epithelium regions in comparison to the other montages. The underlying motivation in considering montages involving the forehead was the potential to “force” current flow in a downward trajectory toward the lower return electrodes and thereby targeting the ROI in its path. While there is some current flow in the ROI, the montages comprising the nose bridge electrode clearly hold more promise and were subjected to further analysis. This extended evaluation allowed us to perform a detailed current flow analysis through not only the OE but also other relevant structures of interest (OB, basal ganglia, and the hippocampus).

For each of the three optimal montages, the inventor considered surface EF plots for the whole brain and individually for the OB and OE. The whole brain plots confirmed the expected dominant downward current flow for Montages 2 and 3 given the location of the electrodes. This is shown by the increased current flow through the orbitofrontal cortex and ending at the lower surface of the cerebellum in these two montages. However, Montage 1 showed increased current flow through the orbitofrontal cortex but ending at the occipital lobe (visual cortex). With respect to targeting the olfactory bulb, the electrical current flow modeling virtually demonstrated the potential efficacy of all the three montages to direct the electrical current into the olfactory bulb. In the context of the olfactory epithelium, all of the three montages are also capable of effectively directing current on the anterior ⅓ sub-section of the olfactory epithelium). On the other hand, the inventors observed that Montage 1 led to the most widespread current flow in the ⅓ middle and ⅓ posterior sub-sections of the OE. Montage 3 had moderate effect on the ⅓ posterior sub-section of the OE, whereas Montage 3 demonstrated the least influence on the middle and posterior olfactory epithelium among the three montages.

The consideration of additional potential drivers of polarization along an exemplary axon in each of the three sub-sections in the olfactory epithelium helps in further studying the differences among the optimal montages. Further, all plots were normalized relative to the highest induced value (observed in Montage 1) to enable easier comparison. While the EF component aligned with the axon plots reveals different profiles in each sub-section,, we observed similar profiles across each of the three optimal montages. We also noted a similar pattern when considering the derivative of EF along the axon or the activating function. Each montage indicated a similar profile but the profile was different in different sub-sections. This is likely explained due to the similar electrode configuration employed with all, involving a combination of electrodes positioned at the front (around the nose bridge) with electrodes at the back of the head, resulting in similar overall current flow pattern. This results in a similar voltage profile in any one sub-compartment which naturally manifests into similar polarization metrics as both EF components along the axon and the activating function are related to voltage. The notable difference across montages is the highest induced value for Montage 1 for both EF in the axon direction and the normalized activating function. This makes Montage 1 the ideal candidate when considering the highest likelihood for activation of the OE across all montages considered.

As part of the final detailed analysis, the inventors considered representative coronal 2D slices at three different sections to highlight current flow patterns through the basal ganglia (considering their role in Parkinson’s disease) and particularly the hippocampus/parahippoampus/EC as the secondary terminals of olfactory pathways and considering their reported roles in the pathogenesis of the early stages of Alzheimer’s disease. Our simulations indicate that Montage 1 induced higher EF magnitude overall in the brain—particularly on the cortical structures including motor and somatosensory cortices. On the other hand, Montages 2 and 3 have the least impact over upper segments of the cerebrum and cortical structures including the motor and somatosensory cortices but have more localized and prominent effects over the hippocampal/parahippocampal/EC. For the structures comprising the basal ganglia and internal capsule, Montage 1 was found to have higher induced current flow in comparison to Montages 2 and 3.

In brief, all three montages were capable of stimulation of the olfactory bulb, 1; 3 anterior sub-segment of olfactory epithelium, and orbitofrontal cortex, but due to the dominant downward trajectory of current flow for Montages 2 and 3, higher EF was induced in the hippocampal and parahippocampal/EC cortex and terminated at the cerebellum. However, Montage 1 directed the current to the overall cerebrum with relatively less impact on the hippocampal/parahippocampus/EC and more impact on the motor/sensory cortex (in comparison to Montages 2 and 3) and the current terminating at the occipital cortex but not in the Cerebellum.

In a preferred embodiment, a headgear device that administers Montage 3’s configuration to the wearer’s head is a practical and easy to use option that enables the modulation of the olfactory region of the brain.

In a preferred embodiment, current is delivered to the electrodes from a neuromodulation device comprising a power source, signal generator, processor, and connecting wires. The neuromodulation device is capable of delivering direct or alternating current to a human subject through the electrodes. The current may be cathodal or anodal.

In one embodiment of the invention, stimulation can be synchronised with a particular segment of respiration cycle, preferably at the end of the inspiration phase to mimic the physiological firing of the Olfactory neurons as explained by Jiang et al. (2017).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A device for delivering optimized electrical stimulation the olfactory bulb, olfactory mucosa, olfactory epithelium, and any combination thereof comprising:
non invasive electrodes;
a source of energy for generating an electric field.

2. The device, in accordance with claim 1, wherein the electrodes sit on each side of the nose bridge and on the upper posterior region of the scalp.

3. The device, in accordance with claim 1, wherein the electrodes sit on the nose bridge.

4. The device, in accordance with claim 1, wherein the electrodes side on each side of the nose bridge.

5. The device, in accordance with claim 1, wherein the electrodes sit in the center of the lower forehead, on the nose bridge, and on the neck posterior.

6. The device, in accordance with claim 1, wherein one electrode sits on each side of the nose bridge and two electrodes sit on the upper posterior region of the scalp.

7. The device, in accordance with claim 1, wherein three electrodes sit on the lower forehead, one electrode sits on each side of the nose bridge, and two electrodes sit on the neck posterior.

8. The device, in accordance with claim 1, wherein stimulation is delivered in closed-loop systems with physiological sensors including but not limited to one or more of the following: electromyography, electroencephalography, infra-red sensing, artificial intelligence based image recognition, or electrocardiography.

9. The device, in accordance with claim 1, wherein stimulation is synchronised with a particular segment of respiration cycle, such as at the end of the inspiration phase to mimic the physiological firing of the olfactory neurons.

10. A device for delivering optimized electrical stimulation to the hippocampal and parahippocampal cortex comprising;
non invasive electrodes sitting on the center of the lower forehead, on the nose bridge, and on the neck posterior,
a source of energy for generating an electric field.

11. The device, in accordance with claim 10, wherein one electrode sits on each side of the nose bridge and two electrodes sit on the upper posterior region of the scalp.

12. The device, in accordance with claim 10, wherein three electrodes sit on the lower forehead, one electrode sits on each side of the nose bridge, and two electrodes sit on the neck posterior.

13. A device for delivering optimized electrical stimulation to the olfactory region of a brain comprising:
a frame configured to be worn on the head of a user, the frame including a bridge configured to be supported on the nose of the user with two nose bridge electrodes;
a forehead portion coupled to and extending away from the bridge with a lower forehead electrode centered between the wearers eyebrows, and 2 arms coupled to the brow portion and extending around the wearer’s ears with neck posterior electrodes attached at the ends , the arms being configured to be positioned over the temple of the user with the free ends disposed behind the ears of the user, wherein the bridge is adjustable for selective positioning.

14. The device, in accordance with claim 13, wherein the first arm houses the control unit.

15. The device, in accordance with claim 13, wherein the forehead portion houses a camera.

16. A method for delivering optimized electrical stimulation to the olfactory bulb, olfactory mucosa, olfactory epithelium, and any combination thereof comprising;
placing non invasive electrodes on the face and scalp of the patient;
delivering an electric current to the electrodes.

17. The method, in accordance with claim 16, wherein the electrodes sit on each side of the nose bridge and on the upper posterior region of the patient’s scalp.

18. The method, in accordance with claim 16, wherein the electrodes sit in the center of the patient’s lower forehead, on the patient’s nose bridge, and on the patient’s neck posterior.

19. The method, in accordance with claim 16, wherein one electrode sits on each side of the patient’s nose bridge and two electrodes sit on the upper posterior region of the patient’s scalp.

20. The method, in accordance with claim 16, wherein three electrodes sit on the patient’s lower forehead, one electrode sits on each side of the nose bridge, and two electrodes sit on the patient’s neck posterior.

21. A method for delivering optimized electrical stimulation to the hippocampal and parahippocampal cortex comprising:
non invasive electrodes sitting on the center of the lower forehead, on the nose bridge, and on the neck posterior,
delivering an electric current to the electrodes.

22. The device, in accordance with claim 21, wherein one electrode sits on each side of the nose bridge and two electrodes sit on the upper posterior region of the scalp.

23. The device, in accordance with claim 21, wherein three electrodes sit on the lower forehead, one electrode sits on each side of the nose bridge, and two electrodes sit on the neck posterior.
